SEQUENCE LISTING (2 sequences)

SEQ ID NO: 1 - DNA, 2425 bp, Homo sapien
ggccgccgagcgcctggaaggagctgctggacaggccgagggagcctccgcccgagaccgcgcagccgccgccgccatgggtaaggcgagcgggccgaaaccgaacgctcggccctcagtcccgggctgagggccgcggccaggcggggtggactgggagctgggggagacgccgaggccgccagtcggaactggggcccgaacccaggatgggcggggactgcctgggactcgggggctcccggggtcgtcatggcaacgcgccccctcccttgttccggcccgttcccgcccgggttctgaggcatagtggccgagggctcgaggtgccacggcgtccaggagcgaggacagggccagctacaaaagaccccacagctgtagagagagcaaacttgttaaacatggctaaactgagtatcaaaggactcattgaatctgctctgagctttggccgcactttggattctgactatccccccttgcagcaattctttgttgttatggaacattgcctgaaacacggtcttaaagtaagaaaatcatttttgagttacaacaaaaccatctggggccctttggaactggtggagaagctgtaccccgaagcagaggaaataggagctagtgtccgggatctacctggtctgaagacccctctgggtcgagcaagagcgtggcttcgattagccctcatgcaaaaaaaaatggccgattacttacgttgcttaattattcagagggatctcttgagtgagttttatgagtatcacgcactaatgatggaagaagaaggagcagtaattgttgggctgctggttggcctgaatgtgatcgatgctaatctgtgtgtgaagggagaggatttagactcacaagttggagtgattgatttttctatgtatttaaagaatgaagaagatattggaaataaagaaaggaatgttcaaattgctgccatattagaccaaaagaattatgttgaagaattaaatagacaactgaacagcacagtcagcagcctccattcaagagttgattcattagaaaagtcaaatactaagctgattgaagagttagcaatagcaaagaataacatcattaaactccaggaagaaaatcatcaattacgaagtgaaaataaattgattttaatgaaaacacagcagcacctagaggttaccaaagtagatgtggaaactgagcttcaaacatataagcattctcgtcaggggctagatgaaatgtacaatgaagccagaaggcagcttcgagatgaatctcagttacgacaggatgtagagaatgagctagcagtacaagttagtatgaagcatgagattgaacttgccatgaagttgctggagaaagatatccatgagaaacaagatactctgataggccttcgacaacaactagaggaagttaaagcaattaacatagagatgtatcaaaagttgcagggttctgaagatggcttgaaagaaaaaaatgaaataattgcccgactagaagaaaaaaccaataaaattactgcagccatgaggcagctggaacaaagattgcagcaagcagagaaggcgcaaatggaagctgaagatgaggatgagaaatatctacaagaatgtctcagtaaatctgatagtctgcagaaacaaatctcccaaaaggagaaacagctggtgcaactggaaactgacttgaagattgagaaggaatggaggcagactttgcaggaagatcttcaaaaggagaaagatgccttatctcatcttagaaatgagactcaacaaatcattagtcttaaaaaagagttccttaacctccaggatgaaaatcagcagttgaaaaaaatatatcatgaacaagagcaagctcttcaagaactcggcaacaagcttagcgaatcaaaacttaaaattgaagacataaaagaagccaacaaagcattgcagggactggtttggctgaaagacaaagaagcaacacattgtaaactttgtgaaaaggaattctcactctctaagagaaagcaccactgtagaaattgtggggaaattttctgtaatgcctgctctgacaacgaactacctttgccttcttcaccaaaaccagtacgggtttgtgattcctgtcatgcactgctcattcagagatgctcatctaacttgccctgagactccagaactaaatccttatgtatgaaattacctacaatgaatgttatgatgttgtatacaaagacatggcagctactgcatcttcatgtgtcacaaactctacatctgcttctcctgtggctctgccatcagctccaatatcaatatgaactcgtattggatttagtggtgagaagaactaaataaaaggaaacacttatttacttttctattgagaatattaaagtaaattaccacataccttg

SEQ ID NO: 2 - protein, 655 aa, Homo sapien
MGGDCLGLGGSRGRHGNAPPPLFRPVPARVLRHSGRGLEVPRRPGARTGPATKDPTAVERANLLNMAKLSIKGLIESALSFGRTLDSDYPPLQQFFVVMEHCLKHGLKVRKSFLSYNKTIWGPLELVEKLYPEAEEIGASVRDLPGLKTPLGRARAWLRLALMQKKMADYLRCLIIQRDLLSEFYEYHALMMEEEGAVIVGLLVGLNVIDANLCVKGEDLDSQVGVIDFSMYLKNEEDIGNKERNVQIAAILDQKNYVEELNRQLNSTVSSLHSRVDSLEKSNTKLIEELAIAKNNIIKLQEENHQLRSENKLILMKTQQHLEVTKVDVETELQTYKHSRQGLDEMYNEARRQLRDESQLRQDVENELAVQVSMKHEIELAMKLLEKDIHEKQDTLIGLRQQLEEVKAINIEMYQKLQGSEDGLKEKNEIIARLEEKTNKITAAMRQLEQRLQQAEKAQMEAEDEDEKYLQECLSKSDSLQKQISQKEKQLVQLETDLKIEKEWRQTLQEDLQKEKDALSHLRNETQQIISLKKEFLNLQDENQQLKKIYHEQEQALQELGNKLSESKLKIEDIKEANKALQGLVWLKDKEATHCKLCEKEFSLSKRKHHCRNCGEIFCNACSDNELPLPSSPKPVRVCDSCHALLIQRCSSNLP

(19) United States
(12) Patent Application Publication
(10) Pub. No.: US 2010/0041026 A1
(43) Pub. Date: Feb. 18, 2010

(54) Method for Identiflying Modulators of Rufy2 Useful for Treating Alzheimer's Disease

(76) Inventors: John M. Majercak, Wayne, PA (US); William J. Ray, Lansdale, PA (US); David J. Stone, Bothell, WA (US)

Correspondence Address: MERCK AND CO., INC, P O BOX 2000, RAHWAY, NJ, 07065-0907, US

(21) Appl. No.: 11/922,527

(22) PCT Filed: Jun. 23, 2006
(86) PCT No.: PCT/US06/24612

§ 371 Date: Dec. 19, 2007

Related U.S. Application Data
(60) Provisional application No. 60/694,626, filed on Jun. 28, 2005.

(51) Int. Cl. C12Q 1/68; C12Q 1/02

(52) U.S. Cl. 435/6; 435/29

(57) ABSTRACT

Compositions and methods for identifying modulators of RUFY2 are described. The methods are particularly useful for identifying analytes that antagonize RUFY2's effect on processing of amyloid precursor protein to Aβ peptide and thus useful for identifying analytes that can be used for treating Alzheimer disease.

### BACKGROUND OF THE INVENTION

(1) Field of the Invention

The present invention relates to compositions and methods for identifying modulators of RUFY2. The methods are particularly useful for identifying analytes that antagonize RUFY2's effect on processing of amyloid precursor protein to Aβ peptide and thus useful for identifying analytes that can be used for treating Alzheimer disease.

(2) Description of Related Art

Alzheimer's disease is a common, chronic neurodegenerative disease, characterized by a progressive loss of memory and sometimes severe behavioral abnormalities, as well as an impairment of other cognitive functions that often leads to dementia and death. It ranks as the fourth leading cause of death in industrialized societies after heart disease, cancer, and stroke. The incidence of Alzheimer's disease is high, with an estimated 2.5 to 4 million patients affected in the United States and perhaps 17 to 25 million worldwide. Moreover, the number of sufferers is expected to grow as the population ages.

A characteristic feature of Alzheimer's disease is the presence of large numbers of insoluble deposits, known as amyloid plaques, in the brains of those affected. Autopsies have shown that amyloid plaques are found in the brains of virtually all Alzheimer's patients and that the degree of amyloid plaque deposition often correlates with the degree of dementia (Cummings & Cotman, Lancet 326: 1524-1587 (1995)). While some opinion holds that amyloid plaques are a late stage by-product of the disease process, the consensus view is that amyloid plaques and/or soluble aggregates of amyloid peptides are more likely to be intimately, and perhaps causally, involved in Alzheimer's disease.

A variety of experimental evidence supports this view. For example, amyloid β (Aβ) peptide, a primary component of amyloid plaques, is toxic to neurons in culture and transgenic mice that overproduce Aβ peptide in their brains show extensive deposition of Aβ into amyloid plaques as well as significant neuronal toxicity (Yankner, Science 250: 279-282 (1990); Mattson et al., J. Neurosci. 12: 379-389 (1992); Games et al., Nature 373: 523-527 (1995); LaFerla et al., Nature Genetics 9: 21-29 (1995)). Mutations in the APP gene, leading to increased Aβ production, have been linked to heritable forms of Alzheimer's disease (Goate et al., Nature 349:704-706 (1991); Chartier-Harlan et al., Nature 353:844846 (1991); Murrel et al., Science 254: 97-99 (1991); Mullan et al., Nature Genetics 1: 345-347 (1992)). Presenilin-1 (PS1) and presenilin-2 (PS2) related familial early-onset Alzheimer's disease (FAD) shows disproportionately increased production of Aβ1-42, the 42 amino acid isoform of Aβ, as opposed to Aβ1-40, the 40 amino acid isoform (Scheuner et al, Nature Medicine 2: 864-870 (1996)). The longer isoform of Aβ is more prone to aggregation than the shorter isoform (Jarrett et al, Biochemistry 32:4693-4697 (1993). Injection of the insoluble, fibrillar form of Aβ into monkey brains results in the development of pathology (neuronal destruction, tau phosphorylation, microglial proliferation) that closely mimics Alzheimer's disease in humans (Geula et al., Nature Medicine 4:827-831 (1998)). See, Selkoe, J., Neuropathol. Exp. Neurol. 53: 438-447 (1994) for a review of the evidence that amyloid plaques have a central role in Altheimer's disease.

Aβ peptide, a 39-43 amino acid peptide derived by proteolytic cleavage of the amyloid precursor protein (APP), is the major component of amyloid plaques (Glenner and Wong, Biochem. Biophys. Res. Comm. 120: 885-890 (1984)). APP is actually a family of polypeptides produced by alternative splicing from a single gene. Major forms of APP are known as APP695, APP751, and APP770, with the subscripts referring to the number of amino acids in each splice variant (Ponte et al., Nature 331: 525-527 (1988); Tanzi et al., Nature 331: 528-530 (1988); Kitaguchi et al., Nature 331: 530-532(1988)). APP is a ubiquitous membrane-spanning (type 1) glycoprotein that undergoes proteolytic cleavage by at least two pathways (Selkoe, Trends Cell Biol. 8: 447-453 (1998)). In one pathway, cleavage by an enzymee known as α-secretase occurs while APP is still in the trans-Golgi secretory compartment (Kuentzel et al., Biochem. J. 295:367-378 (1993)). This cleavage by α-secretase occurs within the Aβ peptide portion of APP, thus precluding the formation of Aβ peptide. In an alternative proteolytic pathway, cleavage of the Met596-Asp597 bond (numbered according to the 695 amino acid protein) by an enzyme known as β-secretase occurs. This cleavage by β-secretase generates the N-terminus of Aβ peptide. The C-terminus is formed by cleavage by a second enzyme known as γ-secretase. The C-terminus is actually a heterogeneous collection of cleavage sites rather than a single site since γ-secretase activity occurs over a short stretch of APP amino acids rather than at a single peptide bond. Peptides of 40 or 42 amino acids in length (Aβ1-40 and Aβ1-42, respectively) predominate among the C-termini generated by γ-secretase. Aβ1-42 peptide is more prone to aggregation than Aβ1-40 peptide, the major secreted species (Jarrett et al., Biochemistry 32: 4693-4697 91993); Kuo et al., J. Biol. Chem. 271: 4077-4081 (1996)), and its production is closely associated with the development of Alzheimer's disease (Sinha and Lieberburg, Proc. Natl. Acad. Sci. USA 96: 11049-11053 (1999)). The bond cleaved by γ-secretase appears to be situated within the transmembrane domain of APP. For a review that discusses APP and its processing, see Selkoe, Trends Cell. Biol. 8: 447-453 (1998).

While abundant evidence suggests that extracellular accumulation and deposition of Aβ peptide is a central event in the etiology of Alzheimer's disease, recent studies have also proposed that increased intracellular accumulation of Aβ peptide or amyloid containing C-terminal fragments may play a role in the pathophysiology of Alzheimer's disease. For example, over-expression of APP harboring mutations which cause familial Alzheimer's disease results in the increased intracellular accumulation of C99, the carboxy-terminal 99 amino acids of APP containing Aβ peptide, in neuronal cultures and Aβ42 in BEK 293 cells in neuronal cultures and Aβ42 peptide in HEK 293 cells. Moreover, evidence suggests that intra- and extracellular Aβ peptide are formed in distinct cellular pools in hippocampal neurons and that a common feature associated with two types of familial Alzheimer's disease mutations in APP (“Swedish” and “London”) is an increased intracellular accumulation of Aβ42 peptide. Thus, based on these studies and earlier reports implicating extracellular Aβ peptide accumulation in Alzheimer's disease pathology, it appears that altered APP catabolism may be involved in disease progression.

Much interest has focused on the possibility of inhibiting the development of amyloid plaques as a means of preventing or ameliorating the symptoms of Alzheimer's disease. To that end, a promising strategy is to inhibit the activity of β- and γ-secretase, the two enzymes that together are responsible for producing Aβ. This strategy is attractive because, if the formation of amyloid plaques is a result of the deposition of Aβ is a cause of Alzeimer's disease, inhibiting the activity of one or both of the two secretases would intervene in the disease process at an early stage, before late-stage events such as inflammation or apoptosis occur. Such early stage intervention is expected to be particularly beneficial (see, for example, Citron, Molecular Medicine Today 6:392-397 (2000)).

To that end, various assays have been developed that are directed to the identification of substances that may interfere with the production of Aβ peptide or its deposition into amyloid plaques. U.S. Pat. No. 5,441,870 is directed to methods of monitoring the processing of APP by detecting the production of amino terminal fragments of APP. U.S. Pat. No. 5,605,811 is directed to methods of identifying inhibitors of the production of amino terminal fragments of APP. U.S. Pat. No. 5,593,846 is directed to methods of detecting soluble Aβ by the use of binding substances such as antibodies. US Published Patent Application No. US20030200555 describes using amyloid precursor proteins with modified β-secretase cleavage sites to monitor beta-secretase activity. Esler et al., Nature Biotechnology 15: 258-263 (1997) described an assay that monitored the deposition of Aβ peptide from solution onto a synthetic analogue of an amyloid plaque. The assay was suitable for identifying substances that could inhibit the deposition of Aβ peptide. However, this assay is not suitable for identifying substances, such as inhibitors of β- or γ-secretase, that would prevent the formation of Aβ peptide.

Various groups have cloned and sequenced cDNA encoding a protein believed to be β-secretase (Vassar et al., Science 286: 735-741 (1999); Hussain et al., Mol. Cell. Neurosci. 14: 419-427 (1999); Yan et al., Nature 402: 533-537 (1999); Sinha et al., Nature 402: 537-540 (1999); Lin et al., Proc. Natl. Acad. Sci. USA 97: 1456-1460 (2000)). U.S. Pat. Nos. 6,828,117 and 6,737,510 disclose a secretase, which the inventors call aspartyl protease 2 (Asp2), variant Asp-2(a) and variant Asp-2(b), respectively, and U.S. Pat. No. 6,545,127 discloses a catalytically active enzyme known as memapsin. Hong et al., Science 290: 150-153 (2000) determined the crystal structure of the protease domain of human β-secretase complexed with an eight-residue peptide-like inhibitor at 1.9 angstrom resolution. Compared to other human aspartic proteases, the active site of human β-secretase is more open and less hydrophobic, contributing to the broad substrate specificity of human β-secretase (Lin et al., Proc. Natl. Acad. Sci. USA 97: 1456-1460 (2000)).

Ghosh et al., J. Am. Chem. Soc. 122: 3522-3523 (2000) disclosed two inhibitors of β-secretase, OM99-1 and OM99-2, that are modified peptides based on the β-secretase cleavage site of the Swedish mutation of APP (SEVNL/DAEFR, with “/” indicating the site of cleavage). OM99-1 has the structure VNL*AAEF (with “L*A” indicating the uncleavable hydroxyethylene transition-state isostere of the LA peptide bond) and exhibits a Ki towards recombinant β-secretase produced in E. coli of 6.84×10⁻⁸ M±2.72×10⁻⁹ M. OM99-2 has the structure EVNL*AAEF (with “L*A” indicating the uncleavable hydroxyethylene transition-state isostere of the LA peptide bond) and exhibits a Ki towards recombinant β-secretase produced in E. coli of 9.58×10⁻⁹ M±2.86×10⁻¹⁰ M. OM99-1 and OM99-2, as well as related substances, are described in International Patent Publication WO0100665.

Currently, most drug discovery programs for Alzheimer's disease have targeted either aceytlcholinesterase or the secretase proteins directly responsible for APP processing. While acetylcholinesterase inhibitors are marketed drugs for Alzheimer's disease, they have limited efficacy and do not have disease modifying properties. Secretase inhibitors, on the other hand, have been plagued either by mechanism-based toxicity (γ-secretase inhibitors) or by extreme difficulties in identifying small molecule inhibitors with appropriate pharmacokinetic properties to allow them to become drugs 03ACE inhibitors). Identifying novel factors involved in APP processing would expand the range of targets for Alzheimer's disease treatments and therapy.

### BRIEF SUMMARY OF THE INVENTION

The present invention provides compositions and methods for identifying modulators of RUFY2. The methods are particularly useful for identifying analytes that antagonize RUFY2's effect on processing of amyloid precursor protein to Aβ peptide and thus useful for identifying analytes that can be used for treating Alzheimer disease.

Therefore in one embodiment the present invention provides a nucleotide sequence (SEQ ID NO:1) of an isolated human cDNA encoding a human RUFY2 polypeptide as shown in SEQ ID NO:2. RUFY2 was identified in a screen of an siRNA library as set forth in Example 1.

In another embodiment, the present invention provides a method for screening for analytes that antagonize processing of amyloid precursor protein (APP) to Aβ peptide, comprising providing recombinant cells, which ectopically expresses RUFY2 and the APP; incubating the cells in a culture medium under conditions for expression of the RUFY2 and APP and which contains an analyte; removing the culture medium from the recombinant cells; and determining the amount of at least one processing product of APP selected from the group consisting of sAPPβ and Aβ peptide in the medium wherein a decrease in the amount of the processing product in the medium compared to the amount of the processing product in medium from recombinant cells incubated in medium without the analyte indicates that the analyte is an antagonist of the processing of the APP to Aβ peptide.

In further aspects of the method, the recombinant cells each comprises a first nucleic acid that encodes RUFY2 operably linked to a first heterologous promoter and a second nucleic acid that encodes an APP operably linked to a second heterologous promoter. In preferred aspects of the present invention, the APP is APP_{NFEV}. In preferred aspects, the method includes a control which comprises providing recombinant cells that ectopically express the APP but not the RUFY2.

The present invention further provides a method for screening for analytes that antagonize processing of amyloid precursor protein (APP) to amyloid β (Aβ) peptide, comprising providing recombinant cells, which ectopically express RUFY2 and a recombinant APP comprising APP fused to a transcription factor that when removed from the APP during processing of the APP produces an active transcription factor, and a reporter gene operably linked to a promoter inducible by the transcription factor; incubating the cells in a culture medium under conditions for expression of the RUFY2 and recombinant APP and which contains an analyte; and determining expression of the reporter gene wherein a decrease in expression of the reporter gene compared to expression of the reporter gene in recombinant cells in a culture medium without the analyte indicates that the analyte is an antagonist of the processing of the APP to Aβ peptide.

In further aspects of the method, the recombinant cells each comprises a first nucleic acid that encodes RUFY2 operably linked to a first heterologous promoter, a second nucleic acid that encodes the recombinant APP operably linked to a second heterologous promoter, and a third nucleic acid that encodes a reporter gene operably linked to promoter responsive to the transcription factor comprising the recombinant APP.

In light of the analytes that can be identified using the above methods, the present invention further provides a method for treating Alzheimer's disease in an individual which comprises providing to the individual an effective amount of an antagonist of RUFY2 activity.

Further still, the present invention provides a method for identifying an individual who has Alzheimer's disease or is at risk of developing Alzheimer's disease comprising obtaining a sample from the individual and measuring the amount of RUFY2 in the sample.

Further still, the present invention provides for the use of an antagonist of RUFY2 for the manufacture of a medicament for the treatment of Alzheimer's disease.

Further still, the present invention provides for the use of an antibody specific for RUFY2 for the manufacture of a medicament for the treatment of Alzheimer's disease.

Further still, the present invention provides a vaccine for preventing and/or treating Alzheimer's disease in a subject, comprising an antibody raised against an antigenic amount of RUFY2 wherein the antibody antagonizes the processing of APP to Aβ peptide.

The term “analyte” refers to a compound, chemical, agent, composition, antibody, peptide, aptamer, nucleic acid, or the like, which can modulate the activity of RUFY2.

The term “RUFY2” refers to one of the genes from the RUFY gene family from a human, mouse or other mammal, whose human nucleotide and amino acid sequences are given in FIGS. 1 and 2, respectively. The gene family known as RUFY refers to a gene family designated as the RUN and FYVE domain-containing (RUFY) protein family which has been shown to be a downstream affector of Etk. The RUN domain is associated with interactions between the RUN-containing protein and a small GTPase signaling molecule such as one of the Rab proteins (Callebaut, et al., Trends Biochem Sci. 26(2):79-83 (2001)). Rabs generally control the trafficking of vesicles throughout cells. RUFY2 also contains a FYVE domain, a sequence motif found predominantly in vesicle associated proteins (Stenmark, et al., J. Biol. Chem. 271: 24048-24054 (1996)). The protein sequence is identical to the protein product of Genbank ID number NP_{—}060457. The nucleotide sequence is identical to the sequence reported as Genbank D number NM_{—}017987. The term further includes mutants, variants, alleles, and polymorphs of RUFY2. Where appropriate, the term further includes fusion proteins comprising all or a portion of the amino acid sequence of RUFY2 fused to the amino acid sequence of a heterologous peptide or polypeptide, for example, hybrid immuoglobulins comprising the amino acid sequence, or domains thereof, of RUFY2 fused at its C-terminus to the N-terminus of an immunoglobulin constant region amino acid sequence (see, for example, U.S. Pat. No. 5,428,130 and related patents).

### BRIEF DESCRIPTION OF THE DRAWINGS

FIG. 1 is a nucleic sequence encoding the human RUFY2.

FIG. 2 is the amino acid sequence of the human RUFY2.

FIG. 3 is a graph showing the relative expression of the metabolites expressed as a percent of the mean control non-silencing siRNA value of 100. RUFY2 p<0.05 for EV40, EV42, and ≈0.2 for sAPPβ and p≈0.5 for sAPPα.

FIG. 4 shows the tissue distribution of RUFY2 mRNA in various human tissues.

FIG. 5 shows the localization of RUFY2 to the region of chromosome 10 that harbors a locus associated both with Alzheimer's disease and Aβ levels in patients. Ad loci located on chromosome 10 at or near D10S1225, ( . . . ) Myers et al., Am. J. Med. Genet., 114: 235-244 (2002); (______) Ertekin-Taner et al., Science 290: 2303-2304 (2000); (▾) Curtis et al., Ann. Hum. Genet. 65: 473-482 (2001). The solid vertical bar represents the location of the RUFY2 gene; the X-axis denotes the distance in centimorgans from the Pter on Chromosome 10.

FIG. 6 is a graph showing the reduced secretion of EV40 ad EV42 following RUY2 siRNA transfection of human neuroblastoma SH-SY5Y cells.

FIG. 7 is a graph showing that RUFY2 reduced EV40 in mouse primary neuronal cell culture.

FIG. 8A-8K shows the in situ hybridization of an antisense probe to RUFY2 within regions of the brain.

### DETAILED DESCRIPTION OF THE INVENTION

The protein referred to herein as RUFY2 is a neuronal associated protein that the applicants have discovered to have a role in processing of amyloid precursor protein (APP) to amyloid β (Aβ) peptide. RUFY2 is one member of a gene family designated as the RUN and FYVE domain-containing (RUFY) protein family that has been identified as the downstream effector of Etk (Yang, et al, J. Biol. Chem. 277 (33): 30219-30226 (2002)). Etk has been associated with cellular processes including proliferation, differentiation, motility and apoptosis. Id. The RUFY gene family (RUFY1 and RUFY2) contains an N-terminal RUN domain and a C-terminal FYVE domain with two coiled-coil domains in-between. Id. They appear to be homologues of mouse Rabip4, Cormant et al., Proc. Natl. Acad. Sci. USA 98:1637-1642 (2001). RUFY2, RUFY1, and Rabip4 are membrane associated proteins that function in vesicle transport from the cell surface to endosomes (Cormant et al., Proc. Natl. Acad. Sci. USA 98:1637-1642 (2001), Yang, et al., J. Biol. Chem. 277 (33): 30219-30226 (2002)). Endosomes are the specialized compartments within cells where A□ can be generated (Huse et al., J. Biol. Chem. 275: 33729-37 (2000), Cataldo et al., J. Neurosci. 17(16): 6142-51 (1997), Vasser et al., Science 286: 735-741 (1999), reviewed by Selkoe et al., Ann. N.Y. Acad. Sci. 777: 57-64 (1996)). Thus, RUFY2 is a protein that is involved in the trafficking of vesicles, and their protein cargo, from the cell surface to the endosomes, a process important in the processing of APP to Aβ. These data strengthen the claim that RUFY2 is involved in Alzheimer's disease.

A defining characteristic of Alzheimer's disease (AD) is the deposition of aggregated plaques containing Aβ peptide in the brains of affected individuals. The applicant's discovery that RUFY2 has a role processing APP to APβ peptide suggests that RUFY2 has a role in the progression of Alzheimer's disease in an individual. Therefore, in light of the applicants' discovery, identifying molecules which target activity or expression of RUFY2 would be expected to lead to treatments or therapies for Alzheimer's disease. Expression or activity of RUFY2 may also be useful as a diagnostic marker for identifying individuals who have Alzheimer's disease or are at risk of developing Alzheimer's disease.

The deposition of aggregated plaques containing amyloid β (Aβ) peptide in the brains of individuals affected with Alzheimer's disease is believed to involve the sequential cleavage of APP by two secretase-mediated cleavages to produce Aβ peptide. The first cleavage event is catalyzed by the type I transmembrane aspartyl protease BACE1. BACE1 cleavage of APP at the BACE cleavage site (between amino acids 596 and 597) generates a 596 amino acid soluble N-terminal sAPPβ fragment and a 99 amino acid C-terminal fragment (βCTF) designated C99. Further cleavage of C99 by γ-secretase (a multicomponent membrane complex consisting of at least presenilin, nicastrin, aph1, and pen2) releases the 40 or 42 amino acid Aβ peptide. An alternative, non-amyloidogenic pathway of APP cleavage is catalyzed by α-secretase, which cleaves APP to produce a 613 amino acid soluble sAPPαN-terminal fragment and an 83 amino acid PCTF fragment designated C83. While ongoing drug discovery efforts have focused on identifying antagonists of BACE1 and γ-secretase mediated cleavage of APP, the complicated nature of Alzheimer's disease suggests that efficacious treatments and therapies for Alzheimer's disease might comprise other targets for modulating APP processing. RUFY2 of the present invention is another target for which modulators (in particular, antagonists) of are expected to provide efficacious treatments or therapies for Alzheimer's disease, either alone or in combination with one or more other modulators of APP processing, for example, antagonists selected from the group consisting of BACE1 and γ-secretase.

RUFY2 was identified by screening a siRNA library for siRNA that inhibited APP processing. As described in Example 1, a library of about 15,200 siRNA pools, each targeting a single gene, was transfected individually into recombinant cells ectopically expressing a recombinant APP (APP_{NFEV}). APP_{NFEV} has been described in U.S. Pub. Pat. Appln. No. 20030200555, comprises isoform 1-695 and has a HA, Myc, and FLAG sequences at the amino acid position 289, an optimized β-cleavage site comprising amino acids NFEV, and a K612V mutation. Metabolites of APP_{NFEV} produced during APP BACE1/γ-secretase or a-secretase processing are sAPPβ with NF at the C-terminus, EV40, and EV42 or sAPPα. EV40 and EV42 are unique Aβ40-like and Aβ42-like peptides that contain the glutamic acid and valine substitutions of APP_{NFEV} and sAPPβ and sAPPα each contain the HA, FLAG, and myc sequences. sAPPβ, sAPPα, EV40, and EV42 were detected by an immunodetection method that used antibodies that were specific for the various APP_{NFEV} metabolites. Expression levels were determined relative to a non-silencing siRNA control.

Following two rounds of screening, which consisted of a primary screen done with the entire library of siRNAs and secondary screening of about 1600 siRNAs performed in triplicate repeats, a siRNA designed to target RUFY2 RNA was found to consistently alter processing of APP to sAPPβ, EV40, and EV42. The nucleic acid targeted by the siRNA has sequence identity to the human RUFY2, GenBank accession number NM_{—}017987, which appears to be similar to the sequence reported in Yang et al., J. Biol. Chem. 277 (33): 30219-30226 (2002). Yang et al. report that RUFY2 is ahomologue of RUFY1 and that its expression is relatively restricted and can only be detected in brain, lung and testis (as compared to the more ubiquitous RUFY1) (Yang et al. at 30221). Yang et al. further report that notwithstanding that they are homologues, mouse Rabip4 and human RUFY1/2 are regulated by different mechanisms and that one or more new RUFY family members may remain to be uncovered. Id.

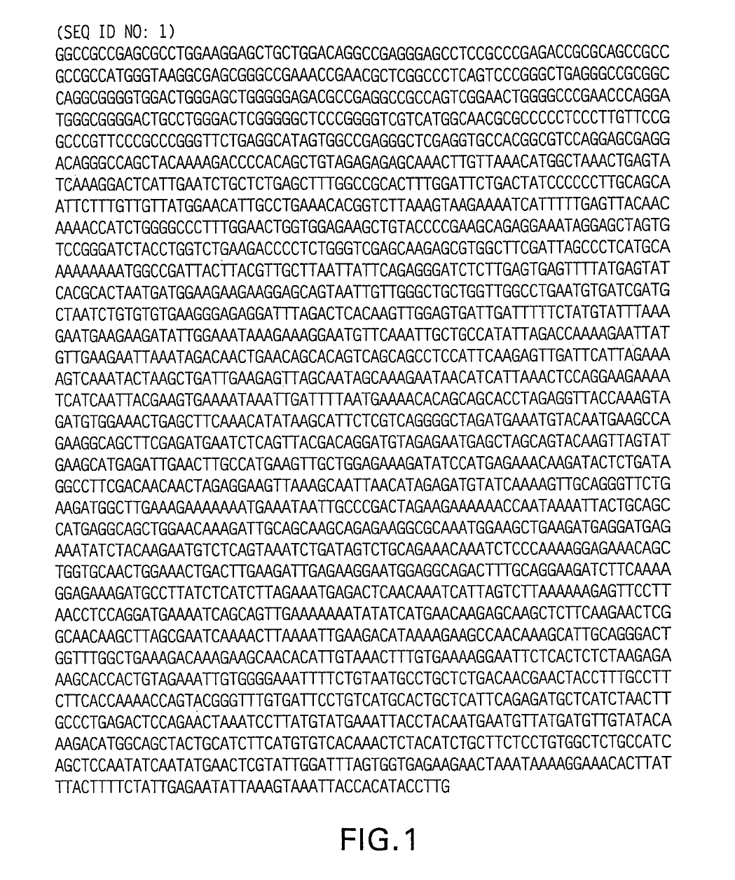

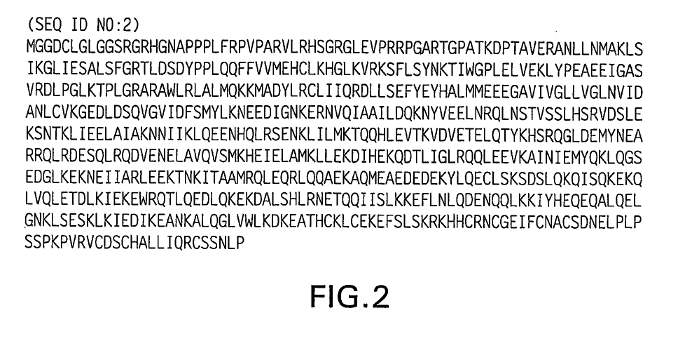

The nucleic acid sequence encoding the human RUFY2 (SEQ ID NO:1) is shown in FIG. 1 and the amino acid sequence for the human RUFY2 (SEQ ID NO:2) is shown in FIG. 2.

The mRNA encoding RUFY2 was found to be preferentially enriched in regions of the brain subject to Alzheimer's disease pathology (Example 2) and the gene encoding RUFY2 resides within a specific region of chromosome 10, a genomic location that has been implicated as harboring genes involved in late onset Alzheimer's disease.

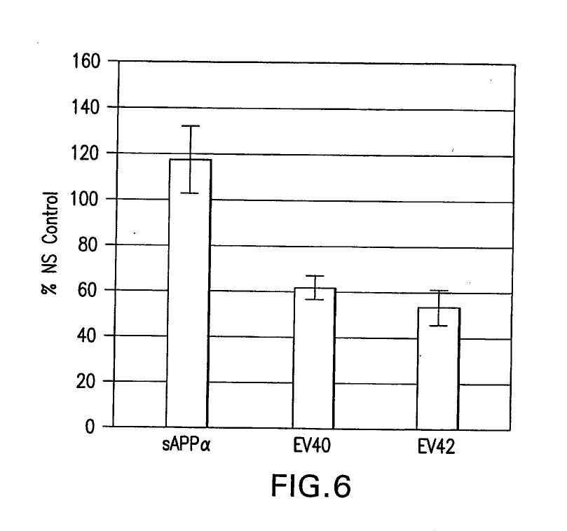

The lowering of EV peptides, as shown in FIG. 6 by the reduced secretion of EV40 and EV42 following si RNA transfection of human neuroblastoma SH-SY5Y cells, suggests that RUFY2 is regulating the production and/or secretion of EV into the conditioned media in a neuronal cell lineage. Similar results are observed transfecting BEK293 NFEV cells with the same RUFY2 siRNAs, but in this instance an ELISA method of APP metabolite detection was used.

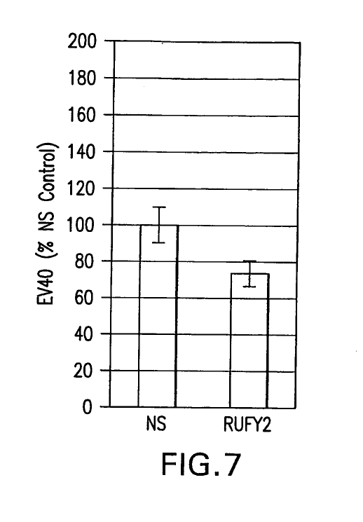

To investigate whether EV40 production can be regulated in neuronal cells within regions of the brain prone to Aβ deposition and plaque pathology, as shown in FIG. 7, mouse primary neurons were co-transfected with APP_{NFEV} cDNA and RUFY2 siRNAs. After five days of RUFY2 knockdown, primary neurons showed a significant (p<0.05) lowering of EV40 suggesting that the amyloid production can be attenuated in neuronal cells prone to Alzheimer's related pathology.

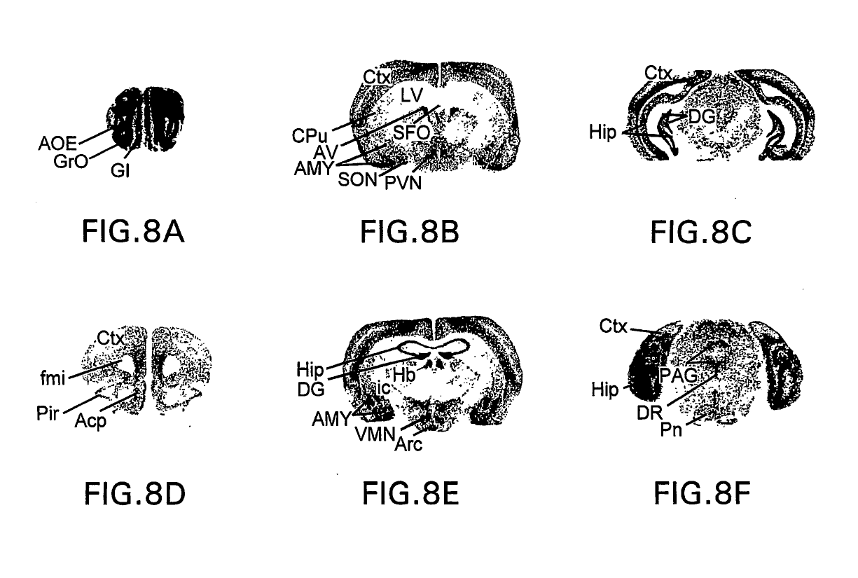

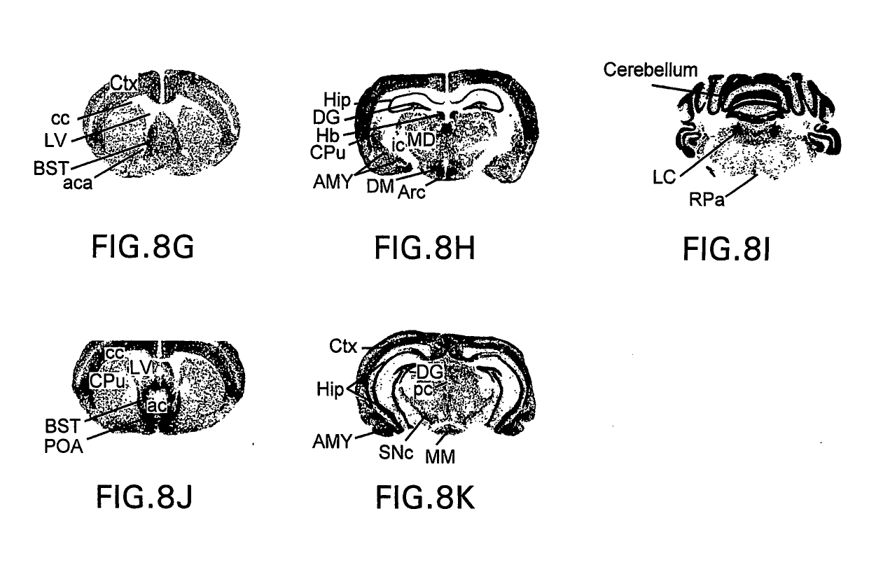

As shown in FIG. 8A-8K, in situ hybridization of an antisense probe to RUFY2 shows prominent expression within many regions of the brain including high level expression within hippocamapal and cortical tissue. The pattern is consistent with neuronal expression within neuronal populations that generate Aβ peptide and suggest that modulation of RUFY2 activity within these cells may alter Alzheimer's disease related pathology.

In light of the applicants' discovery, RUFY2 or modified mutants or variants thereof is useful for identifying analytes which antagonize processing of APP to produce Aβ peptide. These analytes can be used to treat patients afflicted with Alzheimer's disease. RUFY2 can also be used to help diagnose Alzheimer's disease by assessing genetic variability within the locus. RUFY2 can be used alone or in combination with acetylcholinesterase inhibitors, NMDA receptor partial agonists, secretase inhibitors, amyloid-reactive antibodies, growth hormone secretagogues, and other treatments for Alzheimer's disease.

The present invention provides methods for identifying RUFY2 modulators that modulate expression of RUFY2 by contacting RUFY2 with a substance that inhibits or stimulates RUFY2 expression and determining whether expression of RUFY2 polypeptide or nucleic acid molecules encoding an RUFY2 are modified. The present invention also provides methods for identifying modulators that antagonize RUFY2's effect on processing APP to Aβ peptide or formation of AP-amyloid plaques in tissues where RUFY2 is localized or co-expressed. For example, RUFY2 protein can be expressed in cell lines that also express APP and the effect of the modulator on Aβ production is monitored using standard biochemical assays with Aβ-specific antibodies or by mass spectrophotometric techniques. Inhibitors for RUFY2 are identified by screening for a reduction in the release of Aβ peptide which is dependent on the presence of RUFY2 protein for effect. Both small molecules and larger biomolecules that antagonize RUFY2-mediated processing of APP to Aβ peptide can be identified using such an assay. A method for identifying antagonists of RUFY2's effect on the processing APP to Aβ peptide includes the following method which is amenable to high throughput screening. In addition, the methods disclosed in U.S. Pub. Pat. Appln. No. 20030200555 can be adapted to use in assays for identifying antagonists of RUFY2 activity.

A mammalian RUFY2 cDNA, encompassing the first through the last predicted codon contiguously, is amplified from brain total RNA with sequence-specific primers by reverse-transcription polymerase chain reaction (RT-PCR). The amplified sequence is cloned into pcDNA3.zeo or other appropriate mammalian expression vector. Fidelity of the sequence and the ability of the plasmid to encode full-length RUFY2 is validated by DNA sequencing of the RUFY2 plasmid (pcDNA_RUFY2).

Commercially available mammalian expression vectors which are suitable for recombinant RUFY2 expression include, but are not limited to, pcDNA3.neo (Invitrogen, Carlsbad, Calif.), pcDNA3.1 (Invitrogen, Carlsbad, Calif.), pcDNA3.1/Myc-His (Invitrogen), pCI-neo (Promega, Madison, Wis.), pLITMUS28, pLITMUS29, pLITMUS38 and pLITMUS39 (New England Bioloabs, Beverly, Mass.), pcDNAL, pcDNAIamp (Invitrogen), pcDNA3 (Invitrogen), pMC1neo (Stratagene, La Jolla, Calif.), pXT1 (Stratagene), pSG5 (Stratagene), EBO-pSV2-neo (ATCC 37593) pBPV-1(8-2) (ATCC 37110), pdBPV-MMTneo (342-12) (ATCC 37224), pRSVgpt (ATCC 37199), pRSVneo (ATCC 37198), pSV2-dhfr (ATCC 37146), pUCTag (ATCC 37460), 1ZD35 (ATCC 37565), pMC1neo (Stratagene), pcDNA3.1, pCR3.1 (Invitrogen, San Diego, Calif.), EBO-pSV2-neo (ATCC 37593), pCI.neo (Promega), pTRE (Clontech, Palo Alto, Calif.), pV1Jneo, pIRESneo (Clontech, Palo Alto, Calif.), pCEP4 (Invitrogen,), pSC11, and pSV2-dhfr (ATCC 37146). The choice of vector will depend upon the cell type in which it is desired to express the RUFY2, as well as on the level of expression desired, cotransfection with expression vectors encoding APP_{NFEV}, and the like.

Cells transfected with plasmid vector comprising APP_{NFEV}, for example the HBEK293T/APP_{NFEV} cells used to detect RUFY2 activity in the siRNA screening experiment described in Example 1, are used as described in Example 1 with the following modifications. Cells are either cotransfected with a plasmid expression vector comprising APP_{NFEV} operably linked to a heterologous promoter and a plasmid expression vector comprising the RUFY2 operably linked to a heterologous promoter or the HEK²⁹³T/APP_{NFEV} cells described in Example 1 and U.S. Pub. Pat. Appln. 20030200555 are transfected with a plasmid expression vector comprising the RUFY2 operably linked to a heterologous promoter. The promoter comprising the plasmid expression vector can be a constitutive promoter or an inducible promoter. Preferably, the assay includes a negative control comprising the expression vector without the RUFY2.

After the cells have been transfected, the transfected or cotransfected cells are incubated with an analyte being tested for ability to antagonize RUFY2's effect on processing of APP to Aβ peptide. The analyte is assessed for an effect on the RUFY2 transfected or cotransfected cells that is minimal or absent in the negative control cells. In general, the analyte is added to the cell medium the day after the transfection and the cells incubated for one to 24 hours with the analyte. In particular embodiments, the analyte is serially diluted and each dilution provided to a culture of the transfected or cotransfected cells. After the cells have been incubated with the analyte, the medium is removed from the cells and assayed for secreted sAPPα, sAPPβ, EV40, and EV42 as described in Examples 1 and 8. Briefly, the antibodies specific for each of the metabolites is used to detect the metabolites in the medium. Preferably, the cells are assessed for viability.

Analytes that alter the secretion of one or more of EV40, EV42, sAPPα, or sAPPβ in the presence of RUFY2 protein are considered to be modulators of RUFY2 and potentially useful as therapeutic agents for RUFY2-related diseases. Direct inhibition or modulation of RUFY2 can be confirmed using binding assays using the full-length RUFY2, or a domain thereof or a RUFY2 fusion proteins comprising domain(s) coupled to a C-terminal FLAG, or other, epitopes. A cell-free binding assay using full-length RUFY2, or domain(s) thereof or a RUFY2 fusion proteins or membranes containing the RUFY2 integrated therein and labeled-analyte can be performed and the amount of labeled analyte bound to the RUFY2 determined.

The present invention further provides a method for measuring the ability of an analyte to modulate the level of RUFY2 mRNA or protein in a cell. In this method, a cell that expresses RUFY2 is contacted with a candidate compound and the amount of RUFY2 mRNA or protein in the cell is determined. This determination of RUFY2 levels may be made using any of the above-described immunoassays or techniques disclosed herein. The cell can be any RUFY2 expressing cell such as cell transfected with an expression vector comprising RUFY2 operably linked to its native promoter or a cell taken from a brain tissue biopsy from a patient.

The present invention further provides a method of determining whether an individual has a RUFY2-associated disorder or a predisposition for a RUFY2-associated disorder. The method includes providing a tissue or serum sample from an individual and measuring the amount of RUFY2 in the tissue sample. The amount of RUFY2 in the sample is then compared to the amount of RUFY2 in a control sample. An alteration in the amount of RUFY2 in the sample relative to the amount of RUFY2 in the control sample indicates the subject has a RUFY2-associated disorder. A control sample is preferably taken from a matched individual, that is, an individual of similar age, sex, or other general condition but who is not suspected of having a RUFY2 related disorder. In another aspect, the control sample may be taken from the subject at a time when the subject is not suspected of having a condition or disorder associated with abnormal expression of RUFY2.

Other methods for identifying inhibitors of RUFY2 can include blocking the interaction between RUFY2 and the enzymes involved in APP processing or trafficking using standard methodologies for analyzing protein-protein interaction such as fluorescence energy transfer or scintillation proximity assay. Surface Plasmon Resonance can be used to identify molecules that physically interact with purified or recombinant RUFY2.

In accordance with yet another embodiment of the present invention, there are provided antibodies having specific affinity for the RUFY2 or epitope thereof. The term “antibodies” is intended to be a generic term which includes polyclonal antibodies, monoclonal antibodies, Fab fragments, single V_{H} chain antibodies such as those derived from a library of camel or llama antibodies or camelized antibodies (Nuttall et al., Curr. Pharm. Biotechnol. 1: 253-263 (2000); Muyldermans, J. Biotechnol. 74: 277-302 (2001)), and recombinant antibodies. The term “recombinant antibodies” is intended to be a generic term which includes single polypeptide chains comprising the polypeptide sequence of a whole heavy chain antibody or only the amino terminal variable domain of the single heavy chain antibody (V_{H} chain polypeptides) and single polypeptide chains comprising the variable light chain domain (V_{L}) linked to the variable heavy chain domain (V_{H}) to provide a single recombinant polypeptide comprising the Fv region of the antibody molecule (scFv polypeptides) (see Schmiedl et al., J. Immunol. Meth. 242: 101-114 (2000); Schultz et al., Cancer Res. 60: 6663-6669 (2000); Dübel et al., J. Immunol. Meth. 178: 201-209 (1995); and in U.S. Pat. No. 6,207,804 B1 to Huston et al.). Construction of recombinant single V_{H} chain or scFv polypeptides which are specific against an analyte can be obtained using currently available molecular techniques such as phage display (de Haard et al., J. Biol. Chem. 274: 18218-18230 (1999); Saviranta et al., Bioconjugate 9: 725-735 (1999); de Greeff et al., Infect. Immun. 68: 3949-3955 (2000)) or polypeptide synthesis. In further embodiments, the recombinant antibodies include modifications such as polypeptides having particular amino acid residues or ligands or labels such as horseradish peroxidase, alkaline phosphatase, fluors, and the like. Further still embodiments include fusion polypeptides which comprise the above polypeptides fused to a second polypeptide such as a polypeptide comprising protein A or G.

The antibodies specific for RUFY2 can be produced by methods known in the art. For example, polyclonal and monoclonal antibodies can be produced by methods well known in the art, as described, for example, in Harlow and Lane, Antibodies: A Laboratory Manual. Cold Spring Harbor Laboratory Press: Cold Spring Harbor, N.Y. (1988). RUFY2 or fragments thereof can be used as immunogens for generating such antibodies. Alternatively, synthetic peptides can be prepared (using commercially available synthesizers) and used as immunogens. Amino acid sequences can be analyzed by methods well known in the art to determine whether they encode hydrophobic or hydrophilic domains of the corresponding polypeptide. Altered antibodies such as chimeric, humanized, camelized, CDR-grafted, or bifunctional antibodies can also be produced by methods well known in the art. Such antibodies can also be produced by hybridoma, chemical synthesis or recombinant methods described, for example, in Sambrook et al., supra, and Harlow and Lane, supra. Both anti-peptide and anti-fusion protein antibodies can be used (see, for example, Bahouth et al., Trends Pharmacol. Sci. 12: 338 (1991); Ausubel et al., Current Protocols in Molecular Biology, (John Wiley and Sons, N.Y. (1989)).

Antibodies so produced can be used for the immunoaffinity or alfinity chromatography purification of RUFY2 or RUFY2/ligand or analyte complexes. The above referenced anti-RUFY2 antibodies can also be used to modulate the activity of the RUFY2 in living animals, in humans, or in biological tissues isolated therefrom. Accordingly, contemplated herein are compositions comprising a carrier and an amount of an antibody having specificity for RUFY2 effective to block naturally occurring RUFY2 from binding its ligand or for effecting the processing of APP to Aβ peptide.

Therefore, in another aspect, the present invention further provides pharmaceutical compositions that antagonize RUFY2's effect on processing of APP to Aβ peptide. Such compositions include a RUFY2 nucleic acid, RUFY2 peptide, fusion protein comprising RUFY2 or fragment thereof coupled to a heterologous peptide or protein or fragment thereof, an antibody specific for RUFY2, nucleic acid or protein aptamers, siRNA inhibitory to RUFY2 mRNA, analyte that is a RUFY2 antagonist, or combinations thereof, and a pharmaceutically acceptable carrier or diluent.

In a further still aspect, the present invention further provides a kit for in vitro diagnosis of disease by detection of RUFY2 in a biological sample from a patient. A kit for detecting RUFY2 preferably includes a primary antibody capable of binding to RUFY2; and a secondary antibody conjugated to a signal-producing label, the secondary antibody being capable of binding an epitope different from, i.e., spaced from, that to which the primary antibody binds. Such antibodies can be prepared by methods well-known in the art. This kit is most suitable for carrying out a two-antibody sandwich immunoassay, e.g., two-antibody sandwich ELISA.

Using derivatives of RUFY2 protein or cDNA, dominant negative forms of RUFY2 that could interfere with RUFY2-mediated APP processing to Aβ release can be identified. These derivatives could be used in gene therapy strategies or as protein-based therapies top block RUFY2 activity in afflicted patients. RUFY2 can be used to identify endogenous brain proteins that bind to RUFY2 using biochemical purification, genetic interaction, or other techniques common to those skilled in the art. These proteins or their derivatives can subsequently be used to inhibit RUFY2 activity and thus be used to treat Alzheimer's disease. Additionally, polymorphisms in the RUFY2 RNA or in the genomic DNA in and around RUFY2 could be used to diagnose patients at risk for Alzheimer's disease or to identify likely responders in clinical trials.

The following examples are intended to promote a further understanding of the present invention.

### Example 1

RUFY2 was identified in a screen of a siRNA library for modulators of APP processing.

A cell plate was prepared by plating HBEK293T/APP_{NFEV} cells to the wells of a 384-well Corning PDL-coated assay plate at a density of about 2,000 cells per well in 40 μL DMEM containing 10% fetal bovine serum (FBS) and antibiotics. The cell plate was incubated overnight at 37° C. in 5% CO₂. HEK²⁹³T/APP_{NFEV} cells are a subdlone of EEK293T cells stably transformed with the APP_{NFEV} plasmid described in U.S. Pub. Pat. Appl. No. 20030200555. In brief APP_{NFEV} encodes human amyloid precursor protein (APP), isoform 1-695, modified at amino acid position 289 by an in-frame insertion of HA, Myc, and FLAG epitope amino acid sequences and at amino acid positions 595, 596, 597, and 598 by substitution of the amino acid sequence NFEV for the endogenous amino acid sequence KMDA sequence comprising the BACE1 cleavage site. Thus, the BACE cleavage site is a modified BACE1 cleavage site and BACE1 cleaves between amino acids F and E of NFEV. Maintenance of the plasmid within the subclone is achieved by culturing the cells in the presence of the antibiotic puromycin.

The next day, the cells in each of the wells of the cell plate were transfected with a siRNA library as follows. Oligofectamine™ (Invitrogen, Inc., Carlsbad, Calif.) was mixed with Opti-MEM® (Invitrogen, Inc., Carlsbad, Calif.) at a ratio of 1 to 40 and 20 μL of the mixture was added to each well of a different 384-well plate. To each well of the plate, 980 nL of a particular 10 μM siRNA species was added and the plate incubated for ten minutes at room temperature. Afterwards, five μL of each the siRNA/Oligofectamine™/Opti-MEM® mixtures was added to a corresponding well in the cell plate containing the BEK2⁹³/APP_{NFEV} cells. The cell plate was incubated for 24 hours at 37° C. in 5% CO₂. Controls were provided which contained non-silencing siRNA or a siRNA that inhibited BACE 1.

On the next day, for each of the wells of the cell plate, the siRNA and Oligofectamine™/Opti-MEM® mixture was removed and replaced with 70 μL DMEM containing 10% FBS and MERCK compound A (see, WO2003093252, Preparation of spirocyclic [1,2,5]thiadiazole derivatives as γ-secretase inhibitors for treatment of Alzheimer's disease, Collins et al.), a γ-secretase inhibitor given at a final concentration equal to its IC₅₀ in cell-based enzyme assays. The cell plate was incubated for 24 hours at 37° C. in 5% CO₂.

On the next day, for each of the wells of the cell plate, 64 μL of the medium (conditioned medium) was removed and transferred to four 384-well REMP plates in 22, 22, 10, and 10 μL aliquots for subsequent use in detecting sAPPα, EV42, EV40, sAPPβ using AlphaScreen™ (PerkinElmer, Wellesley, Mass.) detection technology. Viability of the cells was determined by adding 40 μL 10% Alamar Blue (Serotec, Inc., Raleigh, N.C.) in DMEM containing 10% FBS to each of the wells of the cell plate with the conditioned medium removed. The cell plate was then incubated at 37° C. for two hours. The Acquest™ (Molecular Devices Corporation, Sunnyvale, Calif.) plate reader was used to assay fluorescence intensity (ex. 545 nm, em. 590 nm) as a means to confirm viability of the cells.

Assays for detecting and measuring sAPPβ, EV42, EV40, and sAPPα were detected using antibodies as follows. In general, detection-specific volumes (8 or 0.5 μL) were transferred to a 384-well white, small-volume detection plate (Greiner Bio-One, Monroe, N.C.). In the case of the smaller volume, 7.5 μL of assay medium was added for a final volume of eight μL per well. One μL of antibody/donor bead mixture (see below) was dispensed into the solution, and one μL antibody/acceptor bead mixture was added. Plates were incubated in the dark for 24 hours at 4° C. Then the plates were read using AlphaQuest™ (PerkinElmer, Wellesley, Mass.) instrumentation. In all protocols, the plating medium was DMEM (Invitrogen, Inc., Carlsbad, Calif.; Cat. No. 21063-029); 10% FBS, the AlphaScreen™ buffer was 50 mM HEPES, 150 mM NaCl, 0.1% BSA, 0.1% Tween-20, pH 7.5, and the AlphaScreen™ Protein A kit was used.

Anti-NF antibodies and anti-EV antibodies were prepared as taught in U.S. Pub. Pat Appln. 20030200555. BACE1 cleaves between amino acids F and E of the NFEV cleavage site of APP_{NFEV} to produce a sAPPβ peptide with NF at the C-terminus and an EV40 or EV42 peptide with amino acids EV at the N-terminus. Anti-NF antibodies bind the C-terminal neoepitope NF at the C-terminus of the sAPPβ peptide produced by BACE1 cleavage of the NFEV sequence of APP_{NFEV}. Anti-EV antibodies bind the N-terminal neoepitope EV at the N-terminus of EV40 and EV42 produced by BACE1cleavage of the NFEV sequence of APP_{NFEV}. Anti-Bio-G2-10 and anti-Bio-G2-11 antibodies are available from the Genetics Company, Zurich, Switzerland. Anti-Bio-G2-11 antibodies bind the neoepitope generated by the γ-secretase cleavage of Aβ or EV peptides at the 42 amino acid position. Anti-Bio-G2-10 antibodies bind the neoepitope generated by the γ-secretase cleavage of Aβ or EV peptides at the 40 amino acid position. Anti-6E10 antibodies are commercially available from Signet Laboratories, Inc., Dedham, Mass. Anti-6E10 antibodies bind the epitope within amino acids 1 to 17 of the N-terminal region of the Aβ and the EV40 and EV42 peptides and also binds sAPPα because the same epitope resides in amino acids 597 to 614 of sAPPα. Bio-M2 anti-FLAG antibodies are available from Sigma-Aldrich, St. Louis, Mo.

Detecting sAPPβ. An AlphaScreen™ assay for detecting sAPPβ-NF produced from cleavage of APP_{NFEV} at the BACE cleavage site was performed as follows. Conditioned medium for each well was diluted 32-fold into a final volume of eight μL. As shown in Table 1, biotinylated-M2 anti-FLAG antibody, which binds the FLAG epitope of the APP_{NFFV}, was captured on streptavidin-coated donor beads by incubating a mixture of the antibody and the streptavidin coated beads for one hour at room temperature in AlphaScreen™ buffer. The amount of antibody was adjusted such that the final concentration of antibody in the detection reaction was 3 nM antibody. Anti-NF antibody was similarly captured separately on protein-A acceptor beads in AlphaScreen™ buffer and used at a final concentration of 1 nM (Table 1). The donor and acceptor beads were each used at final concentrations of 20 μg/mL.

TABLE 1

| Donor/Antibody Bead Mixture | | | Acceptor/Antibody Bead Mixture | | |
|---|---|---|---|---|---|
| | | Final | | | Final |
| | Vol. | Conc. in | | Vol. | Conc. in |
| | (μL) | 50 μL assay | | (μL) | 50 μL assay |
| Anti-Bio-Flag | 1 | 3 nM | NF-IgG | 5 | 1 nM |
| (16 μM) | | | (1.1 μM) | | |
| SA Coated | 23 | 20 μg/mL | Protein A | 23 | 20 μg/mL |
| Donor | | | Acceptor | | |
| Beads | | | Beads | | |
| (5 mg/mL) | | | (5 mg/mL) | | |
| Alpha Buffer | 1131 | | Alpha Buffer | 1127 | |
| Final Vol. | 1155 | | Final Vol. | 1155 | |

Detecting EV42: Conditioned medium for each well was used neat (volume eight μL). As shown in Table 2, anti-Bio-G2-11 antibody was captured on streptavidin-coated donor beads by incubating a mixture of the antibody and the streptavidin coated beads for one hour at room temperature in AlphaScreen™ buffer. The amount of antibody was adjusted such that the final concentration of antibody in the detection reaction was 20 nM antibody. Anti-EV antibody was similarly captured separately on protein-A acceptor beads in AlphaScreen™ buffer and used at a final concentration of 5 nM (Table 2). The donor and acceptor beads were used at final concentrations of 20 μg/mL.

TABLE 2

| | | | Acceptor/Antibody | | |
|---|---|---|---|---|---|
| Donor/Antibody Bead Mixture | | | Bead Mixture | | |
| | | Final | | | Final |
| | Vol. | Conc. in | | Vol. | Conc. in |
| | (μL) | 50 μL assay | | (μL) | 50 μL assay |
| Anti-Bio-G2-11 | 14 | 20 nM | EV-IgG | 23 | 5 nM |
| (8.27 μM) | | | (1.27 μM) | | |
| SA Coated Donor | 23 | 20 μg/mL | Protein A | 23 | 20 μg/mL |
| | | | Acceptor | | |
| Beads (5 mg/mL) | | | Beads | | |
| | | | (5 mg/mL) | | |
| Alpha Buffer | 1118 | | Alpha | 1109 | |
| | | | Buffer | | |
| Final Vol. | 1155 | | Final Vol. | 1155 | |

Detecting EV40: Conditioned medium for each well was diluted four-fold into a final volume eight μL. As shown in Table 3, anti-Bio-G2-10 antibody was captured on streptavidin-coated donor beads by incubating a mixture of the antibody and the streptavidin coated beads for one hour at room temperature in AlphaScreen™ buffer. The amount of antibody was adjusted such that the final concentration of antibody in the detection reaction was 20 nM antibody. Anti-EV antibody was similarly captured separately on protein-A acceptor beads in AlphaScreen™ buffer and used at a final concentration of 5 nM. The donor and acceptor beads were used at final concentrations of 20 μg/mL.

TABLE 3

| | | | Acceptor/Antibody | | |
|---|---|---|---|---|---|
| Donor/Antibody Bead Mixture | | | Bead Mixture | | |
| | | Final | | | Final |
| | Vol. | Conc. in | | Vol. | Conc. in |
| | (μL) | 50 μL assay | | (μL) | 50 μL assay |
| Anti-Bio-G2-10 | 5 | 5 nM | EV-IgG | 23 | 5 nM |
| (6.07 μM) | | | (1.27 μM) | | |
| SA Coated Donor | 23 | 20 μg/mL | Protein A | 23 | 20 μg/mL |
| | | | Acceptor | | |
| Beads (5 mg/mL) | | | Beads | | |
| | | | (5 mg/mL) | | |
| Alpha Buffer | 1127 | | Alpha | 1109 | |
| | | | Buffer | | |
| Final Vol. | 1155 | | Final Vol. | 1155 | |

Detecting sAPPα: Conditioned medium for each well was diluted four-fold into a final volume eight μL. As shown in Table 4, Bio-M2 anti-FLAG antibody was captured on streptavidin-coated donor beads by incubating a mixture of the antibody and the streptavidin coated beads for one hour at room temperature in AlphaScreen™ buffer. Anti-6E10 antibody acceptor beads supplied by the manufacturer (Perkin-Elmer, Inc. makes the beads and conjugates antibody 6E10 to them. Antibody 6E10 is made by Signet Laboratories, Inc.) were used at 30 μg/ml final concentration. The donor beads were used at final concentrations of 20 μg/mL.

TABLE 4

| Donor/Antibody Bead Mixture | | | Acceptor/Antibody Bead Mixture | | |
|---|---|---|---|---|---|
| | | Final | | | Final |
| | Vol. | Conc. in | | Vol. | Conc. in |
| | (μL) | 50 μL assay | | (μL) | 50 μL assay |
| Anti-Bio-Flag | 1 | 5 nM | 6E10-IgG | 34.65 | 30 μg/mL |
| (16 μM) | | | (5 mg/mL) | | |
| SA Coated | 23 | 20 μg/mL | | | |
| Donor | | | | | |
| Beads | | | | | |
| (5 mg/mL) | | | | | |
| Alpha Buffer | 1131 | | Alpha | 1120.35 | |
| | | | Buffer | | |
| Final Vol. | 1155 | | Final Vol. | 1155 | |

About 15,200 single replicate pools of siRNAs were tested for modulation of sAPPβ, sAPPα, EV40 and EV42 by the AlphaScreen™ immunodetection method as described above. Based on the profile from this primary screen, 1,622 siRNA were chosen for an additional round of screening in triplicate. siRNAs were defined as “secretase-like” if a significant decrease in sAPPβ, EV40 and EV42 was detected as well as either no change or an increase in sAPPα.

A siRNA was identified which inhibited an mRNA having a nucleotide sequence encoding a protein which had 100% identity to the nucleotide sequence encoding RUFY2. Compared to control non-silencing siRNAs (set to 100%), RUFY2 siRNA pool significantly decreased EV40 (52.8%), EV42 (48.5%) while increasing sAPPα (120.4%) and decreasing sAPPβ (89.2).

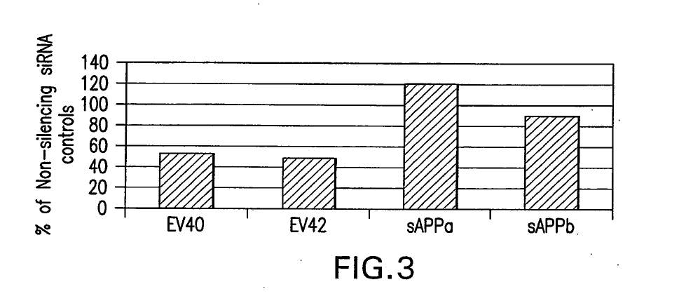

The results are shown schematically in FIG. 3 and show that RUFY2 has a role in APP processing, in particular, the cleavage of APP at the BACE cleavage site, an event necessary in the processing of APP to Aβ peptide. Aβ peptide is a defining characteristic of Alzheimer's disease. Because of its role APP processing, RUFY2 appears to have a role in the establishment or progression of Alzheimer's disease.

### Example 2

Because RUFY2 appeared to have a role in APP processing to Aβ peptide and thus, a role in progression of Alzheimer's disease, expression of RUFY2 was examined in a variety of tissues to determine whether RUFY2 was expressed in the brain.

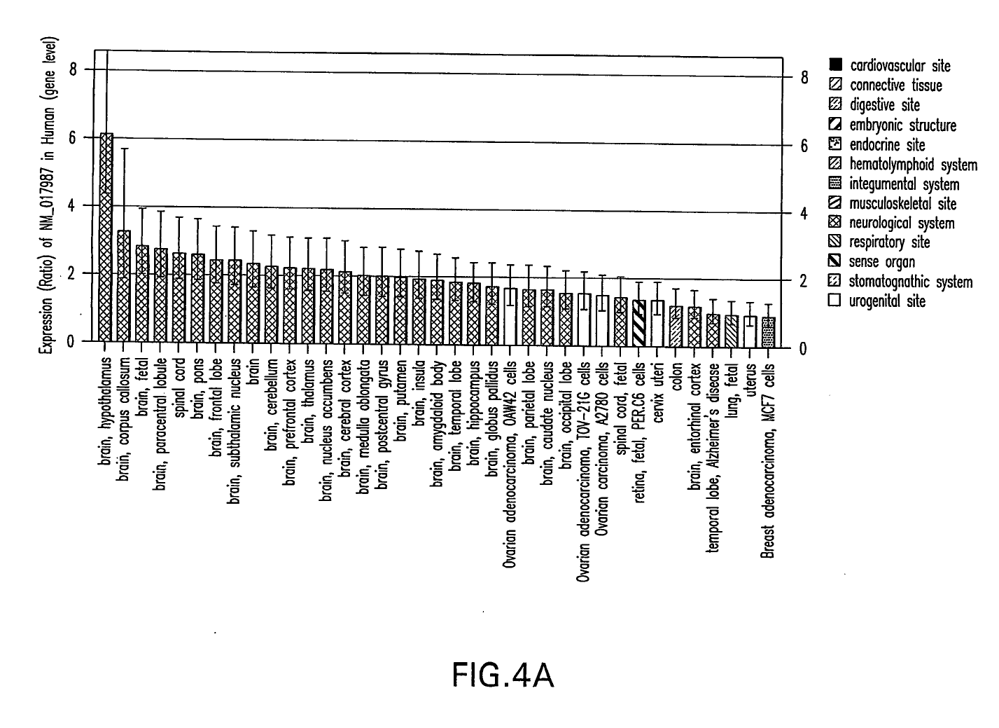

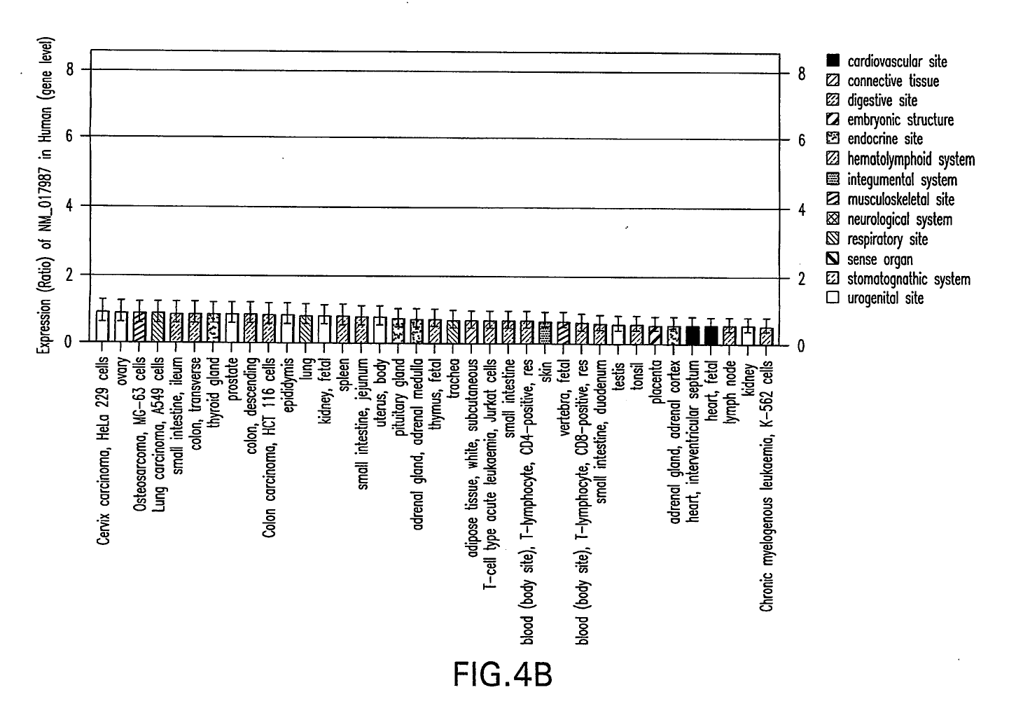

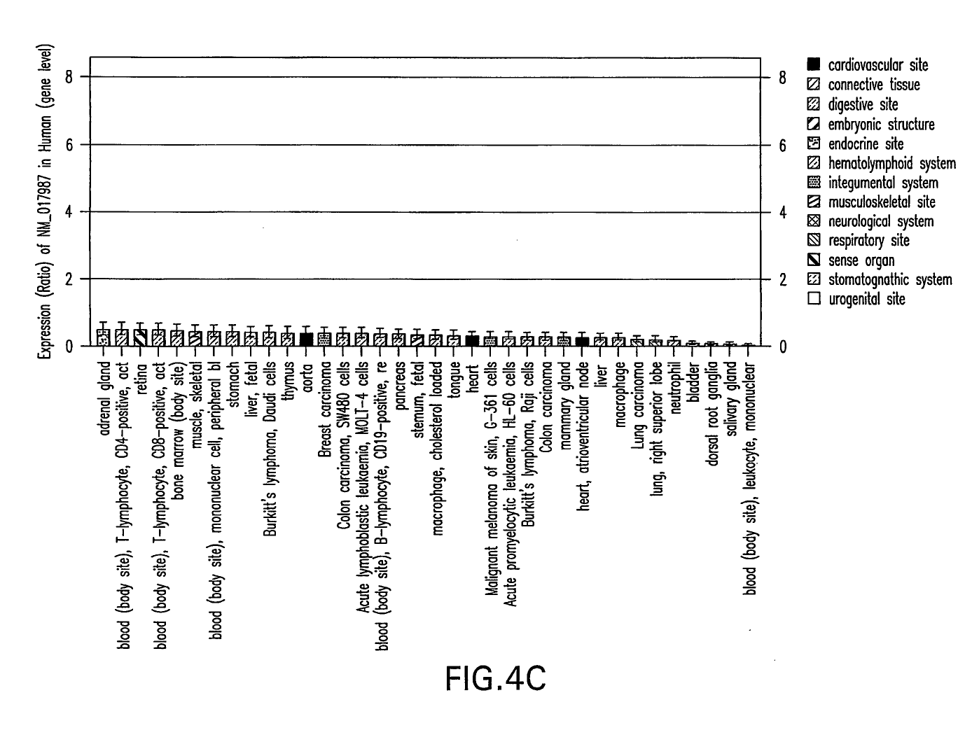

A proprietary database, the TGI Body Atlas, was used to show that the results of a microarray analysis of the expression of a majority of characterized genes, including RUFY2, in the human genome in a panel of different tissues. RUFY2 mRNA was found to be expressed predominantly in the brain and within cortical structures such as the temporal lobe, entorhinal cortex, and prefrontal cortex, all of which are subjected to amyloid Aβ deposition and Alzheimer pathology. The results are summarized in FIG. 4.

The results strengthen the conclusion of the Example 1 that RUFY2 has a role in APP processing and thus, a role in the establishment or progression of Alzheimer's disease.

### Example 3

This example shows that RUFY2 is located within a region of the human genome known to be implicated in late onset of Alzheimer's disease, which further strengthens the conclusion that RUFY2 has a role in the progression of Alzheimer's disease.

Several published population studies have defined genomic locations that influence an individual's propensity to develop Alzheimer's disease. Such studies are able to define particular genomic regions thought to harbor loci that when present or absent, alter an individual chances of developing Alzheimer's disease. The presence of such loci within or near a gene's genomic location is thought to be a strong indicator of that particular gene's potential influence on disease onset or progression. Myers, A., et al., Science 290: 2304-2305 (2000), Ertekin-Taner, et al., Science 290: 2303-2304 (2000) and Kehoe, P., et al., Hum. Mol. Gen. 8 (2): 237-245 (1999) provided evidence suggesting that an Alzheimer's disease locus dependent of the APOE genotype is located on chromosome 10.

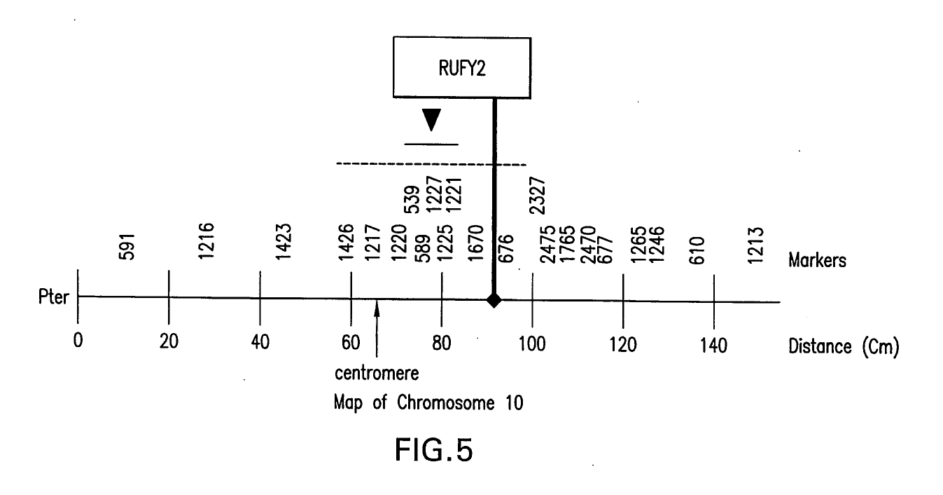

FIG. 5 shows the location of RUFY2 on chromosome 10 relative to the genomic area shown to have linkage to Alzheimer's disease in the above studies. According to public genome numbering convention, RUFY2 is located on chromosome 10 between base pairs 69.7 Mb and 69.9 Mb (10q21.3). This corresponds to a genomic location of about 86 centimorgans (cM) from the Pterminal end (pTer) of chromosome 10. This genomic location falls within a region on chromosome 10 near marker D10S1211, which is a marker of significant linkage to late onset Alzheimer's disease as determined by several independent studies (see, Curtis et al., Annals Hum. Genet., 65: 473-481 (2001)). AD loci located on chromosome 10 at or near D10S1225, ( . . . ) Myers et al., Am. J. Med. Genet., 114: 235-244 (2002); (______) Ertekin-Taner et al., Science 290: 2303-2304 (2000); (▾) Curtis et al., Ann. Hum. Genet. 65: 473-482 (2001) are shown in FIG. 5. The solid vertical line in the middle of the plot is the approximate position of RUFY2. The X axis shows the position of genomic markers (above the X axis) and the distance in centimorgans from pTer (below X-axis).

Thus, RUFY2's close location to the linkage sites identified as being linked to risk for late-onset Alzheimer's disease further supports the conclusion that RUFY2 is risk factor for late-onset Alzheimer's disease and is involved in the establishment or progression of Alzheimer's disease.

### Example 4

SH-SY5Y cells were maintained in 50% DMEM/50% F12, 1× NEAA, 1% pen/strep and 10% FBS prior to transient transfection using an electroporation based procedure of Amaxa corporation (Amaxa, Inc., Gaithersburg, Md.). Following trypsinization cells were counted with a Coulter counter and approximately 2×10⁶ cells per transfection pelleted at low speed (80 g) for ten minutes. Cell pellet was resuspended in 100 μl electroporation buffer (as supplied by Amaxa) with the addition of 2 μg APP_{NFEV} cDNA and 200 μM of a RUFY2 or Non-Silencing (NS) siRNA pool. Cells were pulsed following manufacturers recommended program and seeded into 96 well tissue culture plates for ELISA measurement of secreted APP metabolites following conditioning of the media for 48 hrs. For ELISA, 50 μl of conditioned media plus 50 μl of an alkaline phosphatase (AP) G210 (for EV40 detection), AP-12F4 (for EV42 detection) or AP-P2-1 (for sAPPα detection) was incubated on ELISA plates which had been pre-coated with 6E10 antibody in coating buffer (0.05M carbonate-bicarbonate, pH9.4). Plates were shaken overnight at 4° C. and washed 3× in 0.05% PBST and 2× in AP activation buffer (20 mM Tris, 1 mM MgC12, pH 9.8). Following the incubation in AP substrate (Applied Biosystem#T2214) for 30 minutes, chemiluminescence was measured on a LJL detector. Percent change in sAPPα, EV40 and EV42 levels is represented relative to the Non-Silencing siRNA control.

### Example 5

C57/blk6 mice were housed in our facility (AAALAC certified) in a 12-hour light, 12-hour dark photoperiod with free access to tap water and rodent chow. Post-natal day 1 to day 3 old mice were sacrificed, brains removed and freshly dissociated cortical cells isolated by standard digestion and dissociation procedures. Following isolation, 4×106 cells per transfection were pelleted at low speed for ten minutes. Cell pellet was resuspended in 100 μl electroporation buffer (as supplied by Amaxa) with the addition of 4 μg APP_{NFEV} cDNA and 200 μM of a RUFY2 or Non-Silencing (NS) siRNA pool. Cells were pulsed following manufacturers recommended program and seeded into 6 well tissue culture plates in Neurobasal media supplemented with 1× N2 supplements and 1× Glutamax for five days followed by ELISA measurement of secreted EV40. For ELISA, 50 μl of conditioned media plus 50 μl of a Alkaline phosphatase (AP) G210 was incubated on ELISA plates which had been precoated with 6E10 antibody in coating buffer (0.05M carbonate-bicarbonate, pH9.4). Plates were shaken overnight at 4° C and washed 3× in 0.05% PBST and 2× in AP activation buffer (20 mM Tris, 1 mM MgC12, pH 9.8). Following the incubation in AP substrate (Applied Biosystem#T2214) for 30 minutes, chemiluminescence was measured on a LJL detector. Percent change in EV40 is represented relative to the Non-Silencing siRNA control.

### Example 6

C57/blk6 mice were housed in our facility (AAALAC certified) in a 12-hour light, 12-hour dark photoperiod with free access to tap water and rodent chow. Mice were euthanized, their brains removed and frozen on dry ice and stored at −80° C. 20 μM coronal cryostat sections from adult were hybridized with 6×10⁶ DPM/probe/slide of an antisense or sense ³⁵S-UTP labeled cRNA probe corresponding to nucleotide residues 2011-2415 of SEQ ID NO:1 and opposed to film for five days. The autoradiograms were digitized with a computer-based image analysis system (MCID M5, Imaging Research), processed for brightness/contrast enhancement, and imported into Photoshop (Adobe), where the images were excised from background and anatomical landmarks added for reference (FIG. 8A-8K).

### Example 7

To determine if RUFY2 is a gene linked to Alzheimer's disease and Aβ42 levels on the chromosome 10 q regions, single nucleotide polymorphisms (SMPs) were examined in four independent, case-control AD populations owned by Celera Diagnostics, Alameda, Calif. Briefly, two populations of Alzheimer's patients from the United Kingdom and two from the United Sates of America, comprising approximately 2800 individuals in total, constituted the experimental sample. All AD samples had confined Alzheimer's disease (pre-mortem diagnosis) and the controls were age and gender matched. The APOE genotype was known for all patients. Characteristics for the four cohorts of subjects and controls are shown below in Table 5. In total 2,845 individuals were examined.

TABLE 5

| Sample | Sample Size | Country | AOO or AAE > 75 | ApoE4+ | Female |
|---|---|---|---|---|---|
| Set | (LOAD/Ctrls) | of Origin | (LOAD/Ctrls) | (LOAD/Ctrls) | (LOAD/Ctrls) |
| Cardiff | 392/392 | UK | (214/241) | (223/95) | 301/301 |
| Wash U | 419/375 | USA | (207/200) | (217/81) | 264/235 |
| UCSD | 210/403 | USA | (72/232) | (151/71) | 103/257 |
| UK 2 | 346/308 | UK | (199/233) | (195/77) | 224/196 |

LOAD—late onset Alzheimer's disease;
Crtls = controls;
AOO = age of onset of AD;
AAE = age at examination (when controls were found to be disease free);
ApoE4+ = number of patients that carry at least 1 apoE □4 allele.

Twenty nine SNPs were chosen to cover 360 kb of the human genome, ranging from 63182838-63541936 in the Celera assembly. The SNPs were chosen based upon humanHapMap datato cover the know haplotypes. Population UK2 was used as the exploratory population, and any SNPs that suggested association (p<0.1) with AD in either the entire population or in one of the substrata (gender, age at onset, or apoE □4 genotype) was then examined in the remaining three populations. Results are considered significant if they are p<0.05 in both the UK2 and Meta3 (UK1, WU and SD combined) analysis, or if they are p<0.001 in the meta analysis (all 4 populations combined). Four of the SNPs tested achieved this level of significance, all of which are located roughly in the middle of the genomic area surveyed (63305664-63360759), shown in Table 6 below. Also, all four SNPs showed significance only in the gender substrata (i.e. in males or females only). These SNPs may be of use as biomarkers for prediction of AD in the elderly.

TABLE 6

| | | | | | Allelic Association (UK2 = Discovery Sample) | | | |
|---|---|---|---|---|---|---|---|---|
| | | | | | UK2 | META3 | META | UK1 |
| | | | | | PVALUE | PVALUE | PVALUE | PVALUE |
| Target | Marker | chr | location | Stratification | ASSOC | ASSOC | ASSOC | ASSOC |
| RUFY2 | hCV12038129 | 10 | 63360759 | no | 0.067 | 0.163 | 0.033 | 0.439 |
| RUFY2 | hCV12038129 | 10 | 63360759 | Fem | 0.012 | 0.048 | 0.00267 | 0.436 |
| RUFY2 | hCV12038129 | 10 | 63360759 | Male | 0.778 | 0.722 | 0.619 | 0.038 |
| RUFY2 | hCV16173245 | 10 | 63305664 | no | 0.939 | 0.031 | 0.050 | 1.000 |
| RUFY2 | hCV16173245 | 10 | 63305664 | Fem | 0.709 | 0.974 | 0.824 | 0.378 |
| RUFY2 | hCV16173245 | 10 | 63305664 | Male | 0.343 | 0.00026 | 0.00021 | 0.105 |
| RUFY2 | hCV1058481 | 10 | 63318515 | no | 0.392 | 0.325 | 0.660 | 0.946 |
| RUFY2 | hCV1058481 | 10 | 63318515 | Fem | 0.138 | 0.255 | 0.079 | 0.159 |
| RUFY2 | hCV1058481 | 10 | 63318515 | Male | 0.298 | 0.002 | 0.001 | 0.017 |
| RUFY2 | hCV11596841 | 10 | 63343833 | no | 1.000 | 0.030 | 0.054 | 1.000 |
| RUFY2 | hCV11596841 | 10 | 63343833 | Fem | 1.000 | 0.926 | 0.916 | 0.329 |
| RUFY2 | hCV11596841 | 10 | 63343833 | Male | 0.539 | 0.00029 | 0.00041 | 0.108 |

| | | Allelic Association | | | | | | | |
|---|---|---|---|---|---|---|---|---|---|
| | | (UK2 = Discovery Sample) | | Odds Ratio | | | | | |
| | | SD | WU | UK2 | META3 | META | UK1 | SD | WU |
| | | PVALUE | PVALUE | Odds | odds | odds | odds | odds | odds |
| Target | Marker | ASSOC | ASSOC | ratio | ratio | ratio | ratio | ratio | ratio |
| RUFY2 | hCV12038129 | 0.186 | 0.066 | 1.88 | 1.30 | 1.42 | 0.78 | 1.55 | 1.99 |
| RUFY2 | hCV12038129 | 0.469 | 0.089 | 3.45 | 1.61 | 1.90 | 1.38 | 1.42 | 2.05 |
| RUFY2 | hCV12038129 | 0.357 | 0.503 | 0.79 | 0.89 | 0.87 | 0.26 | 1.61 | 1.69 |
| RUFY2 | hCV16173245 | 0.019 | 0.143 | 0.98 | 0.83 | 0.87 | 1.00 | 0.67 | 0.81 |
| RUFY2 | hCV16173245 | 0.511 | 0.793 | 1.08 | 1.00 | 1.02 | 1.16 | 0.85 | 0.94 |
| RUFY2 | hCV16173245 | 0.013 | 0.038 | 0.75 | 0.58 | 0.61 | 0.60 | 0.53 | 0.61 |
| RUFY2 | hCV1058481 | 0.102 | 0.846 | 0.88 | 1.08 | 1.03 | 0.99 | 1.28 | 1.03 |
| RUFY2 | hCV1058481 | 0.761 | 0.518 | 0.76 | 0.89 | 0.86 | 0.80 | 1.08 | 0.89 |
| RUFY2 | hCV1058481 | 0.088 | 0.190 | 1.36 | 1.54 | 1.50 | 2.01 | 1.50 | 1.35 |
| RUFY2 | hCV11596841 | 0.016 | 0.165 | 1.01 | 1.22 | 1.17 | 1.00 | 1.55 | 1.23 |
| RUFY2 | hCV11596841 | 0.730 | 0.646 | 0.99 | 0.99 | 0.99 | 0.84 | 1.11 | 1.10 |
| RUFY2 | hCV11596841 | 0.004 | 0.115 | 1.24 | 1.79 | 1.66 | 1.79 | 2.23 | 1.50 |

All p-values are 2-sided
Meta 3 . . . : UK1 & WU & SD
Meta . . . : UK2 & UK1 & WU & SD
Meta3 pvalue assoc = p value for the combined UK1, Wash U and San Diego populations;
Meta pvalue assoc = p value for the combined UK1, UK2, Wash U and San Diego populations;
Meta3 odd ratio = odds ratio for the combined UK1, Wash U and San Diego populations;
Meta odds ratio = odds ratio for the combined UK1, UK2, Wash U and San Diego populations.

### Example 8

The results of Examples 1-7 have shown that the RUFY2 has a role in the establishment or progression of Alzheimer's disease. The results suggest that analytes that antagonize RUFY2 activity will be useful for the treatment or therapy of Alzheimner's disease. Therefore, there is a need for assays for identifying analytes that antagonize RUFY2 activity, for example, inhibit binding of RUFY2 to its natural ligand or to BACE1. The following is an assay that can be used to identify analytes that antagonize RUFY2 activity.

HEK293T/APP_{NFEV} cells are transfected with a plasmid encoding the human RUFY2 or a homolog of the human RUFY2, for example, the primate, rodent, or other mammalian RUFY2, using a standard transfection protocols to produce HEK²⁹³T/APP_{NFEV}/RUFY² cells. For example, HEK293T/APP_{NFEV} are plated into a 96-well plate at about 8000 cells per well in 80 μL DMEM containing 10% FBS and antibiotics and the cell plate incubated at 37° C. at 5% CO₂ overnight.

On the next day, a mixture of 600 μL Oligofectamine™ and 3000 μL Opti-MEM® is made and incubated at room temperature for five minutes. Next, 23 μL Opti-MEM is added to each well of a 96-well mixing plate. 50 ng pcDNA_RUFY2 and empty control vector (in 1 μL volume) are added into adjacent wells of the mixing plate in an alternating fashion. The mixing plate is incubated at room temperature for five minutes. Next, 6 μL of the Oligofectamine™ mixture is added to each of the wells of the mixing plate and the mixing plate incubated at room temperature for five minutes. After five minutes, 20 μL of the plasmid/Oligofectamine™ mixture is added to the corresponding well in the plate of HEK293/APP_{NFEV} cells plated in the cell plate and the plates incubated overnight at 37° C. in 5% CO₂.

The next day, the medium is removed from each well and replaced with 100 μL DMEM containing 10% FBS. Analytes being assayed for the ability to antagonize RUFY2-mediated activation of Aβ secretion are added to each well individually. The analytes are assessed for an effect on the APP processing to Aβ peptide in RUFY2 transfected cells that is either minimal or absent in cells transfected with the vector-alone as follows. The cells are incubated at 37° C. at 5% CO₂ overnight The next day, conditioned media is collected the amount of sAPPβ, EV42, EV40, and sAPPα in the conditioned media is determined as described in Example 1. Analytes that effect a decrease in the amounts of sAPPβ, EV42, and EV40 and either an increase or no change in the amount of sAPPα are antagonists of RUFY2. Viability of the cells is determined as in Example 1.

### Example 9

Analytes that alter secretion of EV40, EV42, sAPPα, or sAPPβ only, or more, in the presence of RUFY2 are considered to be modulators of RUFY2 and potential therapeutic agents for treating RUFY2-related diseases. The following is an assay that can be used to confirm direct inhibition or modulation of RUFY2.

To confirm direct inhibition or modulation of RUFY2, RUFY2 is subcloned into expression plasmid vectors such that a fusion protein with C-terminal FLAG epitopes are encoded. These fusion proteins are purified by affinity chromatography, according to manufacturer's instructions, using an ANTI-FLAG M2 agarose resin. RUFY2 fusion proteins are eluted from the ANTI-FLAG column by the addition of FLAG peptide (Asp-Tyr-Lys-Asp-Asp-Asp-Asp-Lys) (Sigma Aldrich, St. Louis, Mo.) resuspended in TBS (50 mM Tris HCl pH 7.4, 150 mM NaCl) to a final concentration of 100 μg/ml. Fractions from the column are collected and concentrations of the fusion proteins determined by A280.

A PD-10 column (Amersham, Boston, Mass.) is used to buffer exchange all eluted fractions containing the RUFY2-fusion proteins and simultaneously remove excess FLAG peptide. The FLAG-RUFY2 fusion proteins are then conjugated to the S series CM5 chip surface (Biacore™ International AB, Uppsala, Sweden) using amine coupling as directed by the manufacturer. A pH scouting protocol is followed to determine the optimal pH conditions for immobilization. Imobilization is conducted at an empirically determined temperature in PBS, pH 7.4, or another similar buffer following a standard Biacore immobilization protocol. The reference spot on the CM5 chip (a non-immobilized surface) serves as background. A third spot on the CM5 chip is conjugated with bovine serum albumin in a similar fashion to serve as a specificity control. Interaction of the putative RUFY2 modulating analyte identified in the assay of Example 5 at various concentrations and RUFY2 are analyzed using the compound characterization wizard on the Biacore S51. Binding experiments are completed at 30° C. using 50 mM Tris pH 7, 200 uM MnC12 or MgC12 (+5% DMSO) or a similar buffer as the running buffer. Prior to each characterization, the instrument is equilibrated three times with assay buffer. Default instructions for characterization are a contact time of 60 seconds, sample injection of 180 seconds and a baseline stabilization of 30 seconds. All solutions are added at a rate of 30 μL/min. Using the BiaEvaluation software (Biacore™ International AB, Uppsala, Sweden), each set of sensorgrams derived from the ligand flowing through the RUFY2-conjugated sensor chip is evaluated and, if binding is observed, an affinity constant determined.

### Example 10

This example describes a method for making polyclonal antibodies specific for the RUFY2 or particular peptide fragments or epitope thereof.

The RUFY2 is produced as described in Example 1 or a peptide fragment comprising a particular amino acid sequence of RUFY2 is synthesized and coupled to a carrier such as BSA or KLH. Antibodies are generated in New Zealand white rabbits over a 1 0-week period. The RUFY2 or peptide fragment or epitope is emulsified by mixing with an equal volume of Freund's complete adjuvant and injected into three subcutaneous dorsal sites for a total of about 0.1 mg RUFY2 per immunization. A booster containing about 0.1 mg RUFY2 or peptide fragment emulsified in an equal volume of Freund's incomplete adjuvant is administered subcutaneously two weeks later. Animals are bled from the articular artery. The blood is allowed to clot and the serum collected by centrifugation. The serum is stored at −20° C.

For purification, the RUFY2 is immobilized on an activated support. Antisera is passed through the sera column and then washed. Specific antibodies are eluted via a pH gradient, collected, and stored in a borate buffer (0.125M total borate) at 0.25 mg/mL. The anti-RUFY2 antibody titers are determined using ELISA methodology with free RUFY2 bound in solid phase (1 pg/well). Detection is obtained using biotinylated anti-rabbit IgG, HRP-SA conjugate, and ABTS.

### Example 11

This example describes a method for making monoclonal antibodies specific for the RUFY2.

BALB/c mice are immunized with an initial injection of about 1 μg of purified RUFY2 per mouse mixed 1:1 with Freund's complete adjuvant. After two weeks, a booster injection of about 1 μg of the antigen is injected into each mouse intravenously without adjuvant. Three days after the booster injection serum from each of the mice is checked for antibodies specific for the RUFY2.

The spleens are removed from mice positive for antibodies specific for the RUFY2 and washed three times with serum-free DMEM and placed in a sterile Petri dish containing about 20 mL of DMEM containing 20% fetal bovine serum, 1 mM pyruvate, 100 units penicillin, and 100 units streptomycin. The cells are released by perfusion with a 23 gauge needle. Afterwards, the cells are pelleted by low-speed centrifugation and the cell pellet is resuspended in 5 mL 0.17 M ammonium chloride and placed on ice for several minutes. Then 5 mL of 20% bovine fetal serum is added and the cells pelleted by low-speed centrifugation. The cells are then resuspended in 10 mL DMEM and mixed with mid-log phase myeloma cells in serum-free DMEM to give a ratio of 3:1. The cell mixture is pelleted by low-speed centrifugation, the supernatant fraction removed, and the pellet allowed to stand for 5 minutes. Next, over a period of 1 minute, 1 mL of 50% polyethylene glycol (PEG) in 0.01 M HEPES, pH 8.1, at 370° C. is added. After 1 minute incubation at 37° C., 1 mL of DMEM is added for a period of another 1 minute, then a third addition of DMEM is added for a further period of 1 minute. Finally, 10 mL of DMEM is added over a period of 2 minutes. Afterwards, the cells are pelleted by low-speed centrifugation and the pellet resuspended in DMEM containing 20% fetal bovine serum, 0.016 mM thymidine, 0.1 hypoxanthine, 0.5 μM aminopterin, and 10% hybridoma cloning factor (HAT medium). The cells are then plated into 96-well plates.

After 3, 5, and 7 days, half the medium in the plates is removed and replaced with fresh HAT medium. After 11 days, the hybridoma cell supernatant is screened by an ELISA assay. In this assay, 96-well plates are coated with the RUFY2. One hundred μL of supernatant from each well is added to a corresponding well on a screening plate and incubated for 1 hour at room temperature. After incubation, each well is washed three times with water and 100 μL of a horseradish peroxide conjugate of goat anti-mouse IgG (H+L), A, M (1:1,500 dilution) is added to each well and incubated for 1 hour at room temperature. Afterwards, the wells are washed three times with water and the substrate OPD/hydrogen peroxide is added and the reaction is allowed to proceed for about 15 minutes at room temperature. Then 100 μL of 1 M HCl is added to stop the reaction and the absorbance of the wells is measured at 490 nm. Cultures that have an absorbance greater than the control wells are removed to two cm² culture dishes, with the addition of normal mouse spleen cells in HAT medium. After a further three days, the cultures are re-screened as above and those that are positive are cloned by limiting dilution. The cells in each two cm² culture dish are counted and the cell concentration adjusted to 1×10⁵ cells per mL. The cells are diluted in complete medium and normal mouse spleen cells are added. The cells are plated in 96-well plates for each dilution. After 10 days, the cells are screened for growth. The growth positive wells are screened for antibody production; those testing positive are expanded to 2 cm² cultures and provided with normal mouse spleen cells. This cloning procedure is repeated until stable antibody producing hybridomas are obtained. The stable hybridomas are progressively expanded to larger culture dishes to provide stocks of the cells.

Production of ascites fluid is performed by injecting intraperitoneally 0.5 mL of pristane into female mice to prime the mice for ascites production. After 10 to 60 days, 4.5×10⁶ cells are injected intraperitoneally into each mouse and ascites fluid is harvested between 7 and 14 days later.

While the present invention is described herein with reference to illustrated embodiments, it should be understood that the invention is not limited hereto. Those having ordinary skill in the art and access to the teachings herein will recognize additional modifications and embodiments within the scope thereof. Therefore, the present invention is limited only by the claims attached herein.

What is claimed:

1. An isolated polynucleotide encoding a RUFY2 polypeptide selected from the group consisting of:
a) a polypeptide comprising the amino acid sequence of SEQ ID NO: 2; and
b) a polypeptide comprising an amino acid sequence at least 95% identical to the amino acid sequence of SEQ ID NO: 2.

2. An isolated polynucleotide of claim 1 comprising SEQ ID NO:1.

3. A probe, vector or recombinant nucleic acid comprising the sequence set forth as SEQ ID NO: 1.

4. An isolated cell comprising the probe, vector or recombinant nucleic acid of claim 3.

5. A method of making an isolated polypeptide comprising the amino acid sequence set forth as SEQ ID NO:2, said method comprising the steps of:
a) introducing the vector or recombinant nucleic acid of claim 4 into a host cell or cellular extract,
b) incubating said host cell or cellular extract under conditions whereby said polypeptide is expressed; and
c) isolating said polypeptide.

6. A method for screening for analytes that antagonize processing of amyloid precursor protein (APP) to Aβ peptide, comprising:
(a) providing recombinant cells, which ectopically expresses RUFY2 and the APP;
(b) incubating the cells in a culture medium under conditions for expression of the RUFY2 and APP and which contains an analyte;
(c) removing the culture medium from the recombinant cells; and
(d) determining the amount of at least one processing product of APP selected from the group consisting of sAPPβ and Aβ peptide in the medium wherein a decrease in the amount of the processing product in the medium compared to the amount of the processing product in medium from recombinant cells incubated in medium without the analyte indicates that the analyte is an antagonist of the processing of the APP to Aβ peptide.

7. The method of claim 6 wherein the recombinant cells each comprises a first nucleic acid that encodes RUFY2 operably linked to a first heterologous promoter and a second nucleic acid that encodes an APP operably linked to a second heterologous promoter.

8. The method of claim 7 wherein the APP is APP_{NFEV}.

9. The method of claim 6 wherein a control is provided which comprises providing recombinant cells which ectopically express the APP but not the RUFY2.

10. A method for screening for analytes that antagonize processing of amyloid precursor protein (APP) to amyloid β (Aβ) peptide, comprising:
(a) providing recombinant cells, which ectopically express RLTFY2 and a recombinant APP comprising APP fused to a transcription factor that when removed from the APP during processing of the APP produces an active transcription factor, and a reporter gene operably linked to a promoter inducible by the transcription factor,
(b) incubating the cells in a culture medium under conditions for expression of the RUFY2 and recombinant APP and which contains an analyte; and
(c) determining expression of the reporter gene wherein a decrease in expression of the reporter gene compared to expression of the reporter gene in recombinant cells in a culture medium without the analyte indicates that the analyte is an antagonist of the processing of the APP to Aβ peptide.

11. A method for treating Alzheimer's disease in an individual comprising providing to the individual an effective amount of an antagonist of RUFY2 activity.

12. A method for identifying an individual who has Alzheimer's disease or is at risk of developing Alzheimer's disease comprising obtaining a sample from the individual and measuring the amount of RUFY2 in the sample.

13. The use of an antagonist of RUFY2 for the manufacture of a medicament for the treatment of Alzheimer's disease.

14. The use of an antibody specific for RUFY2 for the manufacture of a medicament for the treatment of Alzheimer's disease.

15. A vaccine for preventing and/or treating Alzheimer's disease in a subject, comprising an antibody raised against an antigenic amount of RUFY2 wherein the antibody antagonizes the processing of APP to Aβ peptide.
